(19) United States
(12) Patent Application Publication
(10) Pub. No.: US 2014/0252270 A1
(43) Pub. Date: Sep. 11, 2014

(54) PARTICLE-BASED SYSTEMS FOR REMOVAL OF POLLUTANTS FROM GASES AND LIQUIDS

(71) Applicant: SDCmaterials, Inc., Tempe, AZ (US)

(72) Inventor: Stephen Edward LEHMAN, JR., Spartanburg, SC (US)

(21) Appl. No.: 14/198,205

(22) Filed: Mar. 5, 2014

Related U.S. Application Data
(60) Provisional application No. 61/773,550, filed on Mar. 6, 2013.
(60) Provisional application No. 61/780,230, filed on Mar. 13, 2013.

(51) Int. Cl. B01J 20/20; C07C 7/12; C10G 25/00; B01D 53/64

(52) U.S. Cl. CPC B01J 20/20; B01D 53/64; C07C 7/12; C10G 25/003
USPC 252/182.33; 423/210; 422/177; 422/211; 585/823; 208/251 R

(57) ABSTRACT

Systems, compositions, and methods for removing a substance or substances from a material, such as a gas or liquid material, are described. The compositions can comprise composite removal particles. In some embodiments, the composite removal particles can be comprised of support particles made from an inexpensive carrier material, and a reactive particle borne on the support particle. The reactive particle reacts with the substance or substances in the material. The reacted composite removal particles can then be removed from the material, which reduces the amount of the substance or substances present in the material. The composite removal particles are useful for removing pollutants, such as mercury, from exhaust gases, such as flue gas from a power plant combustion unit, and from other materials such as natural gas, liquefied natural gas, fuels, hydrocarbons, petrochemicals, and refinery streams.

### CROSS-REFERENCE TO RELATED APPLICATIONS

This application claims priority benefit of U.S. Provisional Patent Application No. 61/773,550, filed Mar. 6, 2013, and U.S. Provisional Patent Application No. 61/780,230, filed Mar. 13, 2013. The entire contents of those applications are incoporated by reference herein.

### FIELD OF THE INVENTION

The present invention relates generally to systems, compositions, and methods for removal of pollutants, such as mercury, from gases and liquids, such as flue gas, using composite nano-sized/micron-sized particles and other particulate materials.

### BACKGROUND OF THE INVENTION

Coal-burning power plants are a significant worldwide energy source. Approximately 41% of the world electricity supply is generated from coal (see URL www.worldcoal.org/coal/uses-of-coal/coal-electricity). About 45% of the electricity in the United States in 2010 was generated from coal, providing 1.85 trillion kilowatt-hours of energy (U.S. Energy Information Administration, Annual Energy Review 2010).

Coal combustion results in numerous pollutants, and control of these pollutants is essential for public health and the protection of the environment. Mercury is a particularly significant pollutant produced by coal-fired power plants, due to its devastating effects on the human nervous system and its accumulation and long residence time in the environment. Mercury boils at 357° C., and as coal-fired plants operate at much higher temperatures, mercury is emitted in volatile form from coal plants.

Mercury emissions from power plants are generally regulated by governments. The United States has set goals for progressively lower levels of mercury emissions from power plants (Mercury and Air Toxics Standards). Efforts at reducing mercury in flue gas have included injection of activated carbon into the flue gas, wet flue gas desulfurization (wet scrubbers) which removes mercury as well as sulfur, carbon filter beds, depleted brine scrubbing, and selenium filters. Removal of mercury from coal before combustion is also employed. Depending on several factors (quality of coal, other control technologies already present in a power plant, etc.), systems for removal of mercury from flue gas may increase the cost of operating a utility boiler by about 1% to 11% (U.S. Environmental Protection Agency, Mercury Study Report to Congress, Vol. VIII: An Evaluation of Mercury Control Technologies and Costs, 1997).

There is thus a need for additional mercury abatement technologies in order to meet increasingly more stringent regulatory requirements, at a reasonable cost.

### BRIEF SUMMARY OF THE INVENTION

Embodiments of the invention provide for composite removal particles and compositions comprising composite removal particles for removal of substances from a fluid or fluid stream, where the fluid or fluid stream can be a gas, a liquid, a gas stream, or a liquid stream, and systems and methods for using the composite removal particles to remove substances from fluids or fluid streams, that is, liquids, gases, liquid streams, or gas streams.

In one embodiment, the invention embraces composite removal particles, and compositions comprising composite removal particles, wherein a composite removal particle comprises a support particle and a reactive particle. The support particle can be fabricated from a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite, or zeolite. In one embodiment, the support particle is fabricated from silicon dioxide. In another embodiment, the support particle is fabricated from carbon. In one embodiment, the reactive particle can be fabricated from a material such as zinc, gold, silver, tin, magnesium, lead, elemental sulfur, selenium, tellurium, platinum or palladium, or any combination thereof. When the reactive particle is composed of more than one material, the two materials can occur mixed together in reactive particles, or reactive particles of different materials can be affixed to the same support particle, or first composite removal particles comprising support particles bearing reactive particles comprising a first reactive material can be combined with additional composite removal particles comprising support particles bearing reactive particles comprising a different reactive material. In one embodiment, the reactive particle is fabricated from zinc. In another embodiment, the reactive material is fabricated from gold.

In another embodiment, the average diameter of the support particles is between about 250 nm to about 500 microns. In another embodiment, the average diameter of the support particles is between about 500 nm to about 10 microns. In another embodiment, the average diameter of the reactive particles is between about 0.5 nm to about 100 nm. In another embodiment, the average diameter of the reactive particles is between about 1 nm to about 100 nm. In another embodiment, the average diameter of the reactive particles is between about 3 nm to about 20 nm.

In another embodiment, the invention comprises composite reactive particles, further comprising activated carbon mercury abatement material.

In one embodiment, the composite reactive particles are attached to a ceramic or metal structure. In another embodiment, the ceramic or metal structure has a honeycomb structure. In another embodiment, the composite reactive particles are attached to the ceramic or metal structure by a washcoat.

In another embodiment, the invention embraces a system for decreasing the content of mercury in mercury-containing flue gas, comprising composite removal particles or compositions comprising composite removal particles, wherein a composite removal particle comprises a support particle and a reactive particle, and wherein the reactive particle of the composite removal particle combines with mercury in the flue gas, to form a mercury-bearing composite removal particle; and a trap for removal of the mercury-bearing composite removal particles, whereby the mercury content of the flue gas is decreased. In another embodiment of the system, the invention embraces a system for decreasing the content of mercury in mercury-containing flue gas, comprising activated carbon mercury abatement material, and comprising composite removal particles or compositions comprising composite removal particles, wherein a composite removal particle comprises a support particle and a reactive particle, and wherein the reactive particle of the composite removal particle combines with mercury in the flue gas, to form a mercury-bearing composite removal particle, and wherein mercury in the flue gas is adsorbed onto the surface of the activated carbon to form mercury-bearing activated carbon; and a trap for removal of the mercury-bearing composite removal particles and the mercury-bearing activated carbon, whereby the mercury content of the flue gas is decreased.

In another embodiment, the invention embraces a system for decreasing the content of mercury in mercury-containing materials such as natural gas, liquefied natural gas, or other fuels.

In another embodiment, the invention embraces a system for decreasing the content of mercury in mercury-containing materials such as hydrocarbons, petrochemicals, or refinery streams. The mercury-containing material can be in gaseous form or in liquid form. The system comprises composite removal particles or compositions comprising composite removal particles, wherein a composite removal particle comprises a support particle and a reactive particle, and wherein the reactive particle of the composite removal particle combines with mercury in the mercury-containing material, to form a mercury-bearing composite removal particle; and a trap for removal of the mercury-bearing composite removal particles, whereby the mercury content of the mercury-containing material is decreased. In another embodiment of the system, the invention embraces a system for decreasing the content of mercury in mercury-containing materials, comprising activated carbon mercury abatement material, and comprising composite removal particles or compositions comprising composite removal particles, wherein a composite removal particle comprises a support particle and a reactive particle, and wherein the reactive particle of the composite removal particle combines with mercury in the mercury-containing material, to form a mercury-bearing composite removal particle, and wherein mercury in the mercury-containing material is adsorbed onto the surface of the activated carbon to form mercury-bearing activated carbon; and a trap for removal of the mercury-bearing composite removal particles and the mercury-bearing activated carbon, whereby the mercury content of the material is decreased.

The support particle can be fabricated from a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite, or zeolite. In one embodiment, the support particle is fabricated from silicon dioxide. In another embodiment, the support particle is fabricated from carbon. In one embodiment, the reactive particle can be fabricated from a material such as zinc, gold, silver, tin, magnesium, lead, elemental sulfur, selenium, tellurium, platinum or palladium, or a combination thereof. In one embodiment, the reactive particle is fabricated from zinc. In another embodiment, the reactive material is fabricated from gold.

In another embodiment, the average diameter of the support particles is between about 250 nm to about 500 microns. In another embodiment, the average diameter of the support particles is between about 500 nm to about 10 microns. In another embodiment, the average diameter of the reactive particles is between about 0.5 nm to about 100 nm. In another embodiment, the average diameter of the reactive particles is between about 1 nm to about 100 nm. In another embodiment, the average diameter of the reactive particles is between about 3 nm to about 20 nm.

In one embodiment of the system, the composite removal particles are, and are used as, loose bulk composite removal particles. In another embodiment of the system, the composite reactive particles are attached to a ceramic or metal structure, and are used as attached to the structure. In another embodiment of the system, the ceramic or metal structure has a honeycomb structure. In another embodiment of the system, the composite reactive particles are attached to the ceramic or metal structure by a washcoat.

In another embodiment, the invention embraces a method of decreasing the mercury content of mercury-containing flue gas, comprising the steps of contacting the flue gas with composite removal particles, said composite removal particles comprising a support particle and a reactive particle, wherein the reactive particle of the composite removal particle combines with mercury in the flue gas, to form a mercury-bearing composite removal particle; and removing the mercury-bearing composite removal particles from the flue gas. In one embodiment of the method, the step of contacting the flue gas with composite removal particles comprises injecting the composite removal particles into the flue gas. In another embodiment of the method, the step of contacting the flue gas with composite removal particles comprises flowing the flue gas over a support to which the composite removal particles are attached. In another embodiment of the method, the invention embraces use of the composite removal particles, or compositions comprising composite removal particles, with mercury abatement material comprising activated carbon.

In another embodiment, the invention embraces a method of decreasing the mercury content of mercury-containing materials such as natural gas, liquefied natural gas, or other fuels, or of mercury-containing materials such as hydrocarbons, petrochemicals, or refinery streams, comprising the steps of contacting the mercury-containing material with composite removal particles, said composite removal particles comprising a support particle and a reactive particle, wherein the reactive particle of the composite removal particle combines with mercury in the mercury-containing material, to form a mercury-bearing composite removal particle; and removing the mercury-bearing composite removal particles from the material. In one embodiment of the method, the step of contacting the mercury-containing material with composite removal particles comprises injecting the composite removal particles into the mercury-containing material. In another embodiment of the method, the step of contacting the mercury-containing material with composite removal particles comprises flowing the mercury-containing material over a support to which the composite removal particles are attached. In another embodiment of the method, the invention embraces use of the composite removal particles, or compositions comprising composite removal particles, with mercury abatement material comprising activated carbon.

In one embodiment, the invention embraces a kit containing composite removal particles, wherein a composite removal particle comprises a support particle and a reactive particle, and wherein the kit contains sufficient composite removal particles for mercury abatement from flue gas from a power plant, or from mercury-containing materials such as natural gas, liquefied natural gas, or other fuels, or from mercury-containing materials such as hydrocarbons, petrochemicals, or refinery streams. The kit can contain instructions, such as printed materials or computer-readable materials, for use of the composite removal particles in mercury abatement. The support particle can be fabricated from a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite, or zeolite. In one embodiment, the support particle is fabricated from silicon dioxide. In another embodiment, the support particle is fabricated from carbon. In one embodiment, the reactive particle can be fabricated from a material such as zinc, gold, silver, tin, magnesium, lead, elemental sulfur, selenium, tellurium, platinum or palladium, or a combination thereof. In one embodiment, the reactive particle is fabricated from zinc. In another embodiment, the reactive material is fabricated from gold.

In another embodiment, the average diameter of the support particles is between about 250 nm to about 500 microns. In another embodiment, the average diameter of the support particles is between about 500 nm to about 10 microns. In another embodiment, the average diameter of the reactive particles is between about 0.5 nm to about 100 nm. In another embodiment, the average diameter of the reactive particles is between about 1 nm to about 100 nm. In another embodiment, the average diameter of the reactive particles is between about 3 nm to about 20 nm.

In one embodiment, the composite reactive particles are attached to a ceramic or metal structure. In another embodiment, the ceramic or metal structure has a honeycomb structure. In another embodiment, the composite reactive particles are attached to the ceramic or metal structure by a washcoat.

In one embodiment, the invention embraces composite removal particles, wherein a composite removal particle comprises a support particle and a reactive particle, wherein the composite removal particles contain mercury or another pollutant. That is, the composite removal particles have been reacted with mercury to become mercury-bearing composite removal particles, or the composite removal particles have reacted with another pollutant to become pollutant-bearing composite removal particles. The support particle can be fabricated from a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite, or zeolite. In one embodiment, the support particle is fabricated from silicon dioxide. In another embodiment, the support particle is fabricated from carbon. In one embodiment, the reactive particle can be fabricated from a material such as zinc, gold, silver, tin, magnesium, lead, elemental sulfur, selenium, tellurium, platinum or palladium, or a combination thereof, and has reacted with mercury to form a zinc/mercury, gold/mercury, silver/mercury, tin/mercury, magnesium/mercury, lead/mercury, elemental sulfur/mercury, selenium/mercury, tellurium/mercury, platinum/mercury, or palladium/mercury amalgam, or a combination of two or more of zinc, gold, silver, tin, magnesium, lead, elemental sulfur, selenium, tellurium, platinum or palladium with mercury. In one embodiment, the reactive particle is fabricated from zinc, and has reacted with mercury to form a zinc/mercury amalgam. In another embodiment, the reactive material is fabricated from gold and has reacted with mercury to form a gold/mercury amalgam.

In another embodiment, the average diameter of the support particles is between about 250 nm to about 500 microns. In another embodiment, the average diameter of the support particles is between about 500 nm to about 10 microns. In another embodiment, the average diameter of the reactive particles is between about 0.5 nm to about 100 nm. In another embodiment, the average diameter of the reactive particles is between about 1 nm to about 100 nm. In another embodiment, the average diameter of the reactive particles is between about 3 nm to about 20 nm.

In one embodiment, the composite reactive particles are attached to a ceramic or metal structure. In another embodiment, the ceramic or metal structure has a honeycomb structure. In another embodiment, the composite reactive particles are attached to the ceramic or metal structure by a washcoat.

In another embodiment, the invention embraces a concrete extender, comprising mercury-bearing composite removal particles. In another embodiment, the invention embraces a composition comprising concrete, or a concrete mix, wherein the concrete or concrete mix further comprises mercury-bearing composite removal particles.

### BRIEF DESCRIPTION OF THE DRAWINGS

FIG. 1 shows a very simplified schematic of a portion of a coal-burning power plant.

FIG. 2 shows a drawing of one embodiment of the composite removal particle.

FIG. 3 shows a drawing of another embodiment of the composite removal particle, and its interaction with flue gas which contains mercury.

FIG. 4 shows one embodiment of the use of the composite removal particles.

FIG. 5 shows a drawing of another embodiment for using the composite removal particles. FIG. 5A shows the composite removal particle attached to the honeycombs of a monolith (complete monolith not shown). FIG. 5B shows a drawing of another embodiment of the composite removal particle.

FIG. 6 shows another embodiment of the use of the composite removal particles.

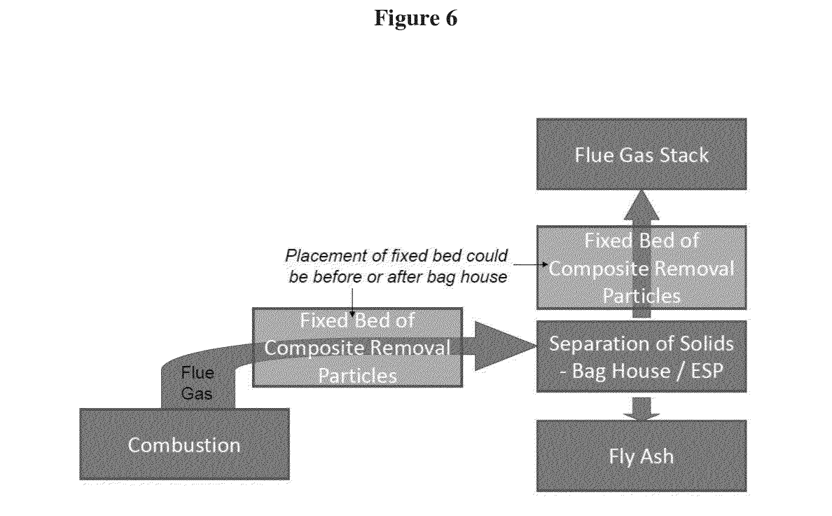

### DETAILED DESCRIPTION OF THE INVENTION

### Definitions

When a number or a numerical range for particle size is given, the number or ranges refer to the average dimension of a collection of particles. As will be appreciated by those skilled in the art, production of particles typically results in a size distribution of particles, which can be characterized by an average dimension (usually particle diameter or particle radius), and a standard deviation. Other useful measures of particle size include ranges which include a certain percentage of particles; for example, a particle distribution may be described by indicating that 90% of the particles in the distribution have diameters between 10 nm and 50 nm.

### Composite Removal Particles

Composite removal particles for use in the invention are typically comprised of a support particle, with one or more reactive material particles attached to the surface of the support particle.

SUPPORT PARTICLES. Support particles for use in the invention are about 250 nm to about 500 microns in diameter, preferably about 500 nm to about 10 microns in diameter. Numerous materials can be used for the support particles. These materials include metal oxides such as iron (II) oxide, iron (III) oxide, mixed iron oxides, copper oxides, titanium dioxide, aluminum oxide, manganese oxide, cerium oxide, molybdenum oxide; metal nitrides such as titanium nitride, molybdenum nitride; metal carbides, such as iron carbide, titanium carbide, molybdenum carbide; carbon; and inorganic oxides and nitrides such as silicon dioxide and silicon carbide. Mixed metal oxide-hydroxides can also be used. Ceramic materials such as boehmite and zeolite can be used as the support particle material.

REACTIVE PARTICLES. The reactive particles are smaller particles which are attached to the surface of the support particle. The reactive particles can be from about 0.5 nm to about 100 nm in diameter, or from about 1 nm to about 100 nm in diameter, preferably from about 3 nm to about 20 nm in diameter. The ratio of the mass of reactive particle material to the mass of support particle material should be about 0.01% to about 30%, preferably about 0.1% to about 5%, more preferably about 1% to about 5%.

When the composite removal particle is intended for use in mercury abatement, the reactive particle material should have good miscibility with mercury and should mix spontaneously with mercury. Examples of reactive particle materials that can be used for mercury abatement include zinc, gold, silver, tin, magnesium, lead, sulfur (elemental sulfur), selenium, and tellurium. Platinum and palladium can also be used, but due to the high price of those metals, they are typically used only when the precious metal can be recovered and recycled, or when the particular application warrants the high expense of using platinum and palladium. Zinc and gold are preferred materials for use as the reactive particle material for mercury abatement, and the preferred size of the reactive particles for mercury abatement is about 3 nm to about 20 nm.

While the preferred configuration of the composite removal particles is the support particle-reactive particle configuration, the composite removal particles may also comprise a single particle made of two or more different materials, where one material is a support material and the other material is a reactive material.

FABRICATION OF COMPOSITE REMOVAL PARTICLES. A wide variety of techniques can be used to prepare the composite removal particles. The particles can be formed by plasma techniques, such as those disclosed in SDCmaterials patents and patent applications U.S. Patent Publication No. 2005/0233380, U.S. Patent Publication No. 2006/0096393, U.S. patent application Ser. No. 12/151,810, U.S. patent application Ser. No. 12/152,084, U.S. patent application Ser. No. 12/151,809, U.S. Pat. No. 7,905,942, U.S. patent application Ser. No. 12/152,111, U.S. Patent Appl. Publication 2008/0280756, U.S. Patent Appl. Publication 2008/0277270, U.S. patent application Ser. No. 12/001,643, U.S. patent application Ser. No. 12/474,081, U.S. patent application Ser. No. 12/001,602, U.S. patent application Ser. No. 12/001,644, U.S. patent application Ser. No. 12/962,518, U.S. patent application Ser. No. 12/962,473, U.S. patent application Ser. No. 12/962,490, U.S. patent application Ser. No. 12/969,264, U.S. patent application Ser. No. 12/962,508, U.S. patent application Ser. No. 12/965,745, U.S. patent application Ser. No. 12/969,503, and U.S. patent application Ser. No. 13/033,514, International Patent Application WO 2011/081834 (PCT/US2010/59763) and U.S. Patent Appl. Publication 2011/0143915 (U.S. patent application Ser. No. 12/962,473). The methods disclosed in U.S. Pat. No. 5,989,648 can also be used. Briefly, a support material and a reactive material are loaded into a plasma gun in a carrier gas. The support and reactive materials are vaporized and/or converted into plasmas, followed by cooling, nucleation, and growth of composite removal particles having a support material portion and a smaller reactive material portion.

Wet chemical techniques and other methods can also be used to create the composite removal particles. Examples of such methods are found in U.S. Pat. No. 6,716,525, U.S. Pat. No. 6,491,985 and U.S. Pat. No. 5,993,988.

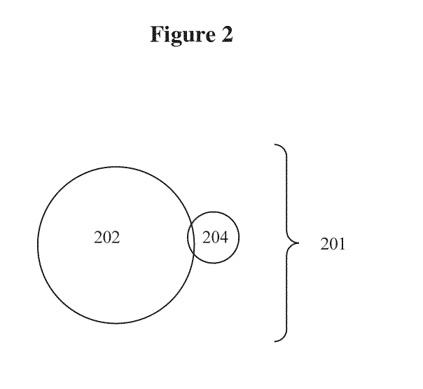

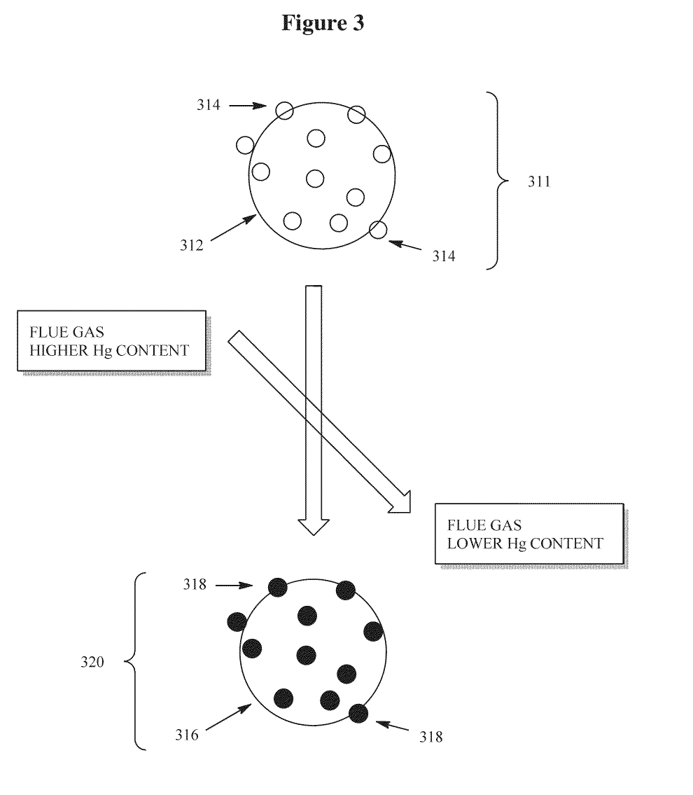

An example of a composite removal particle 201 is shown in FIG. 2. Composite removal particle 201 comprises reactive particle 204, which is borne on the surface of support particle 202. (In all of the Figures, the sizes of the support particle and reactive particle(s) are not necessarily drawn to scale.) A support particle can carry one reactive particle as in FIG. 2, or can carry multiple reactive particles, as shown in FIG. 3. In FIG. 3, the composite removal particle 311 is composed of support particle 312 and multiple reactive particles 314 (only two of the multiple reactive particles are labeled).

When composite removal particle 311 is exposed to a mercury-containing flue gas, the reactive particles 314 absorb mercury from the flue gas, and become mercury-bearing reactive particles 318, affixed to support particle 316. Support particle 316 itself may be essentially unchanged, or may adsorb mercury or other components from the flue gas. After exposure to the flue gas, support particle 316 and reactive particles 318 together form mercury-bearing composite removal particle 320.

Substances for Removal from Gas or Liquid

The composite removal particles can be used for removal of one or several substances from a gas or liquid, or a gas stream or liquid stream.

One particular substance of interest is mercury, which is a common contaminant of flue gas. Flue gas refers to the mixture of gases resulting from combustion in a furnace. The flue gas of coal-burning power plants usually contains a significant amount of mercury, as mercury occurs naturally in coal deposits. For example, coal deposits in the United States have been found to have mercury content ranging from 0.07 parts per million in coal from the Uinta region to 0.24 ppm in the northern Appalachian region (United States Geological Survey, “Mercury in U.S. Coal-Abundance, Distribution, and Modes of Occurrence,” USGS Fact Sheet FS-095-01, September 2001).

Mercury can also be present in the wastewater streams from various industrial processes, for example, in wastewater from chlor-alkali plants using mercury cells. Industrial wastewater streams containing mercury can be treated with the composite removal particles of the invention to remove the mercury before the wastewater is discharged into the environment.

### Treatment of Gas and Liquid Streams and Use of Composite Removal Particles

The composite removal particles, systems, and methods can be used to treat a gas or liquid in order to remove one or more substances from the gas or liquid. Typically, the gas to be treated with the particles and system is an exhaust combustion gas, such as a flue gas, and the substance is a pollutant. The gas stream can also originate from a medical incinerator. The gas stream can also originate from a crematorium. The composite removal particles, systems, and methods can also be used in other industrial processes involving mercury. In one embodiment, the gas is a flue gas, the substance is mercury, and the composite removal particles are used to remove the mercury from the flue gas. In other embodiments, the particles, systems, and methods can be used to treat mercury-containing materials such as natural gas, liquefied natural gas, or other fuels, or mercury-containing materials such as hydrocarbons, petrochemicals, or refinery streams.

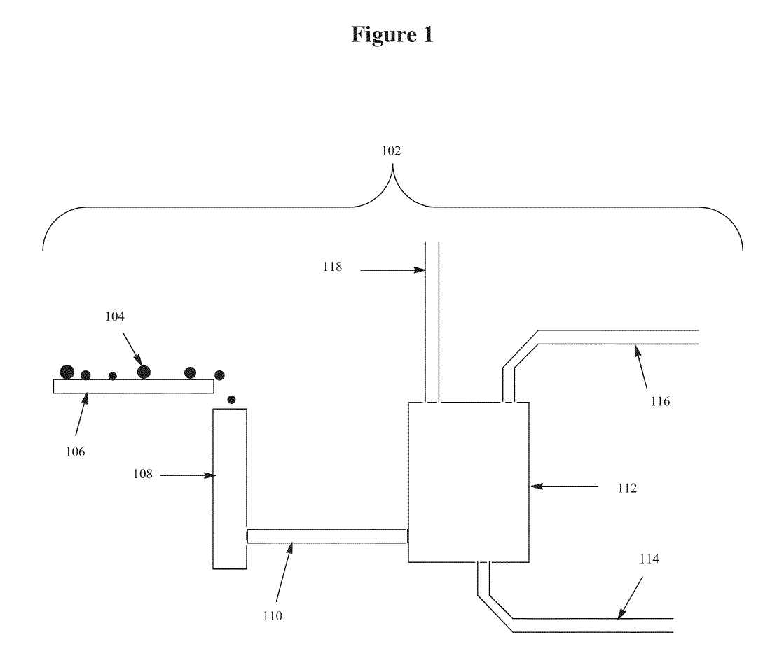

FIG. 1 shows a simplified schematic diagram of a coal-burning furnace and its accessories 102. Pieces of coal 104 are carried by conveyor belt 106 and placed into pulverizer/grinder 108. The pulverized coal is sent through conduit 110 into furnace/boiler 112. Water is heated into steam and carried by conduit 116 to an electrical generator (not shown). Solid ash is collected via conduit 114 for safe disposal. Exhaust gases—that is, flue gases—exit the furnace/boiler through conduit 118 for pollution abatement prior to release into the environment.

Removal of undesired components from gases is often carried out by an overall system comprised of multiple individual systems, where the individual systems can be used sequentially or simultaneously on a single gas stream. For example, flue gas may pass through a unit that removes or decreases sulfur and sulfur oxides, a unit that removes or decreases nitrogen oxides, a unit that removes or decreases mercury, and a unit that removes or decreases fly ash. The composite removal particles can be used at any stage in a gas treatment process. In one embodiment, when used for mercury abatement in flue gas, the composite removal particles are used after the sulfur content of the flue gas has been decreased significantly (sulfur in flue gas is typically in the form of SO₂, SO₃, and H₂SO₄). For example, the sulfur content can be decreased by at least about 50%, at least about 80%, at least about 90%, at least about 95%, or at least about 99%, prior to using the composite removal particles for mercury abatement. In another embodiment, the composite removal particles are used before the sulfur content of the flue gas has been decreased significantly.

When used for mercury removal in flue gas, the composite removal particles can be injected into the flue gas stream, in a continuous or batch process. The particles should be injected at a point in the stream where the temperature of the gas is below the melting point of the particles, and below the melting points of the component support particles or reactive particles. The particles should also be injected at a point in the stream where the temperature of the gas does not cause appreciable coalescence of multiple reactive particles on individual support particles, which would decrease the surface area available to react with mercury (appropriate temperature ranges can be determined by heating the particles, and then examining them using electron microscopy or other methods, in order to identify temperature ranges where the reactive particles do not coalesce). The particles should also be injected at a point in the stream where the temperature of the gas does not significantly decrease the solubility of mercury in the material used as the reactive particles of the composite removal particles (suitable temperatures can be ascertained by consulting a phase diagram for solid solutions of mercury with the material used for the reactive particles). The particles can be entrained in a fluid carrier stream; the fluid carrier can be either another gas (e.g., atmospheric gas) or a liquid (e.g., water). In one embodiment, prior to injection, the particles and the carrier are pre-heated to a temperature of plus or minus about 20% of the temperature of the flue gas at the point of injection, plus or minus about 10% of the temperature of the flue gas at the point of injection, or plus or minus about 5% of the temperature of the flue gas at the point of injection.

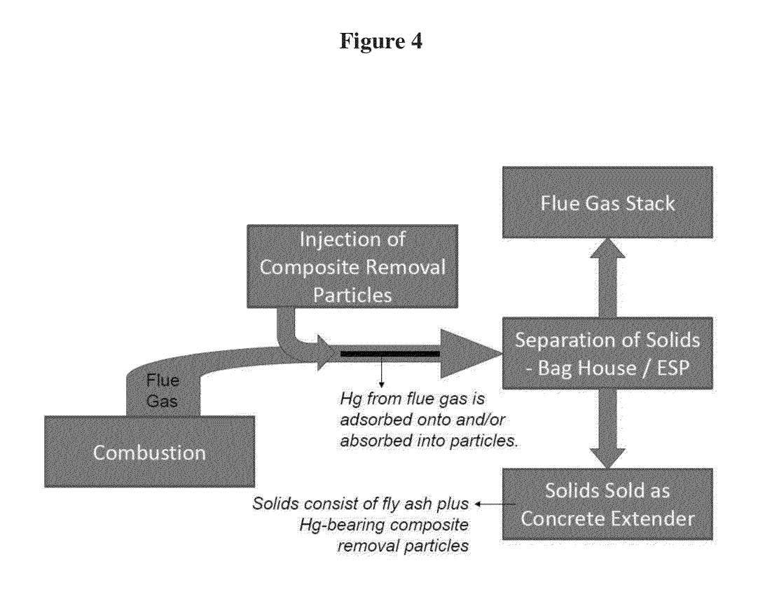

In another embodiment, prior to injection, the particles and the carrier are pre-heated to a temperature of plus or minus about 30° C. of the temperature of the flue gas at the point of injection, plus or minus about 20° C. of the temperature of the flue gas at the point of injection, or plus or minus about 10° C. of the temperature of the flue gas at the point of injection. The injected composite removal particles mix with the flue gas, which contains volatile mercury, and the mercury reacts with the reactive particle component of the composite removal particle. The composite removal particle thus becomes a mercury-bearing composite removal particle. While not wishing to be bound by theory, one possible mechanism by which the reactive particle reacts with the mercury is by formation of an amalgam, that is, formation of an alloy of mercury with another metal. For example, if a composite removal particle having a silicon dioxide support particle and a gold reactive particle is used, after reaction of the gold reactive particle with the mercury in the flue gas, the mercury-bearing composite removal particle will be composed of a silicon dioxide support particle and a mercury-gold amalgam particle. Thus, compared to the practice of injecting activated carbon into the flue gas stream, where mercury is adsorbed onto the carbon particles, the invention absorbs mercury into the reactive particle, leading to greater removal of mercury from the flue gas. FIG. 4 shows a schematic diagram of injection of injection of the composite removal particles into a flue gas stream, in the context of a combustion system.

At a later point in the flue gas stream, the mercury-bearing composite removal particles are removed by a trap, such as filters, cyclones, scrubbing units, a bag house, or an electrostatic precipitator. The trap may be the same apparatus as that used to remove other solids, such as fly ash, from the flue gas. Alternatively, the trap may be a separate apparatus from the fly ash removal apparatus. If, for example, a fly ash removal apparatus removes fly ash from the flue gas prior to injection of the composite removal particles, then a second trap will be necessary to remove the mercury-bearing composite removal particles after treatment of the flue gas with the particles.

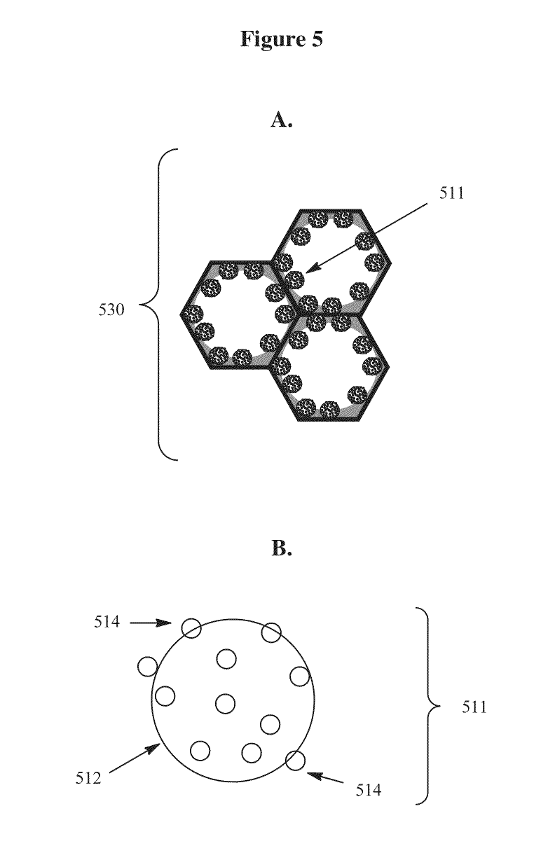

The composite removal particles can also be affixed to a support. The support can be a honeycomb ceramic structure or monolith, or a honeycomb metallic structure or monolith. A washcoat can be used to affix the composite materials to the support. FIG. 5A shows an expanded view of a honeycomb structure, with composite removal particles 511 affixed via a washcoat. Washcoats for attachment of particles to structures are well-known in the art, for example, for affixing ceramic particles to monoliths in catalytic converters, and any standard method can be used to affix the particles to the structure. FIG. 5B shows an expanded view of a single composite removal particle from FIG. 5A. Composite removal particle 511 is composed of support particle 512 and multiple reactive particles 514 (only one of the multiple reactive particles is labeled). The flue gas is passed over the honeycomb structure or monolith. Mercury in the flue gas reacts with the reactive particle portion of the composite removal particles, decreasing the amount of mercury present in the flue gas. The mercury content of the flue gas after it passes over the monolith can be monitored, to indicate when the composite removal particles are saturated with mercury and the monolith needs to be replaced or re-coated with fresh composite removal particles.

The mercury from the mercury-bearing composite removal particles can be recovered by vacuum distillation of the mercury, such as in a standard mercury retort used in industry. This is particularly useful when the reactive particle material is gold or another expensive metal, as recovery of the mercury also leads to recovery of the reactive particle material, which can then be recycled. Alternatively, if recovery of the mercury and reactive particle material is not desired, the solid material removed from the stream can be used as a concrete extender, as is currently done with fly ash removed from flue gas, and as is also done with the solid products recovered following activated carbon injection for mercury abatement. The mercury-bearing composite removal particles can be combined with a concrete mix, or poured together with concrete, to form a concrete or concrete mix having mercury-bearing composite removal particles.

Removal of mercury or other pollutants from natural gas, liquefied natural gas, other fuels, hydrocarbons, petrochemicals, and refinery streams can be accomplished in a similar manner to that described above for removal of mercury from flue gas. In one embodiment, the invention embraces systems and methods for removal of mercury from natural gas (in its gaseous form) or liquefied natural gas (i.e., natural gas compressed and/or cooled to the liquid state). The primary component of natural gas is methane (approximately 70-90%), and natural gas also may contain ethane, propane, and butane (0-20%), carbon dioxide (0-8%), oxygen (0-0.2%), nitrogen (0-5%), hydrogen sulfide (0-5%), and traces of other gases (see URL World-Wide-Web.naturalgas.org/overview/background.asp).

### Efficacy of Mercury Removal

The composite removal particles have the potential to remove much more mercury (or other substances) from the gas or liquid to be treated, compared to existing systems and methods. For example, treatment of flue gas with activated carbon leads to mercury adsorption on the surface of the carbon, while treatment of flue gas with the composite removal particles leads to mercury absorption throughout the volume of the reactive particle, as well as the potential for adsorption on the surface of the particles.

With a loading of reactive particle material of 0.5 to 5% on the support particles, and assuming 0.5 to 5% Hg absorbed in metal, the resulting loading of the mercury-bearing composite removal particles can be 0.0025% to 0.25% (w/w) Hg. Assuming a range of 0.07 to 0.24 ppm Hg in coal yields a range of 0.000028 grams to 0.0096 grams of composite removal particles required for mercury removal per gram of coal burned. In contrast, for activated carbon, about 0.00021 grams to 0.0043 grams of carbon per gram of coal burned are required for mercury removal (90% removal).

The ability of the composite removal particles to absorb mercury, instead of or in addition to adsorbing mercury, also holds the potential for a greater percentage of mercury removal—for example, removal of greater than about 90% of mercury, of greater than about 95% of mercury, of greater than about 98% of mercury, or of greater than about 99% of mercury, with respect to the mercury content of the original gas stream. Additionally, an activated carbon surface adsorbs a very wide variety of materials, while the composite removal particles are much more selective towards mercury removal.

### EXEMPLARY EMBODIMENTS

The invention is further described by the following embodiments.

### Embodiment 1

A system for decreasing the content of mercury in a mercury-containing flue gas stream, comprising composite removal particles positioned in a path of the mercury-containing flue gas stream, wherein a composite removal particle comprises a support particle and a reactive particle, wherein the reactive particle of the composite removal particle combines with mercury in the flue gas stream, to form a mercury-bearing composite removal particle; and a trap for removal of the mercury-bearing composite removal particles, wherein the mercury content of the flue gas stream is decreased.

### Embodiment 2

The system of embodiment 1, wherein the support particle comprises a material selected from the group consisting of a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite and zeolite.

### Embodiment 3

The system of embodiment 2, wherein the support particle comprises silicon dioxide.

### Embodiment 4

The system of any of embodiments 1-3, wherein the reactive particle comprises a material selected from the group consisting of zinc, gold, silver, tin, magnesium, lead, elemental sulfur, selenium, tellurium, platinum, and palladium.

### Embodiment 5

The system of embodiment 4, wherein the reactive particle comprises zinc.

### Embodiment 6

The system of embodiment 4, wherein the reactive particle comprises gold.

### Embodiment 7

The system of any of embodiments 1-6, wherein the average diameter of the support particles is between 250 nm to 500 microns.

### Embodiment 8

The system of any of embodiments 1-6, wherein the average diameter of the support particles is between 500 nm to 10 microns.

### Embodiment 9

The system of any of embodiments 1-8, wherein the average diameter of the reactive particles is between 0.5 nm to 100 nm.

### Embodiment 10

The system of any of embodiments 1-8, wherein the average diameter of the reactive particles is between 3 nm to 20 nm.

### Embodiment 11

The system of any of embodiments 1-10, further comprising a support structure to which the composite removal particles are attached.

### Embodiment 12

The system o of any of embodiments 1-11, further comprising activated carbon mercury abatement material positioned in the path of the flue gas stream.

### Embodiment 13

A method of decreasing the mercury content of mercury-containing flue gas stream, comprising the steps of contacting the flue gas stream with composite removal particles, said composite removal particles comprising a support particle and a reactive particle, wherein the reactive particle of the composite removal particle combines with mercury in the flue gas, to form a mercury-bearing composite removal particle; and removing the mercury-bearing composite removal particles from the flue gas.

### Embodiment 14

The method of embodiment 13, wherein the step of contacting the flue gas with composite removal particles comprises injecting the composite removal particles into the flue gas.

### Embodiment 15

The method of embodiment 13, wherein the step of contacting the flue gas with composite removal particles comprises flowing the flue gas over a support to which the composite removal particles are attached.

### Embodiment 16

The method of any of embodiments 13-15, further comprising, before or after any step, contacting the flue gas with activated carbon.

### Embodiment 17

A composition comprising concrete or a concrete mix, said concrete or concrete mix further comprising mercury-bearing composite removal particles.

### Embodiment 18

A system for decreasing the content of mercury in a material, comprising composite removal particles, wherein a composite removal particle comprises a support particle and a reactive particle, wherein the reactive particle of the composite removal particle combines with mercury in the material, to form a mercury-bearing composite removal particle; and a trap for removal of the mercury-bearing composite removal particles, whereby the mercury content of the material is decreased.

### Embodiment 19

The system of embodiment 18, wherein the material is selected from the group consisting of natural gas, liquefied natural gas, fuels, hydrocarbons, petrochemicals, and refinery streams.

### Embodiment 20

The system of embodiment 19, wherein the material is natural gas.

### Embodiment 21

The system of any of embodiments 18-20, wherein the support particle comprises a material selected from the group consisting of a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite and zeolite.

### Embodiment 22

The system of any of embodiments 18-20, wherein the support particle comprises silicon dioxide.

### Embodiment 23

The system of any of embodiments 18-22, wherein the reactive particle comprises a material selected from the group consisting of zinc, gold, silver, tin, magnesium, lead, elemental sulfur, selenium, tellurium, platinum, and palladium.

### Embodiment 24

The system of embodiment 23, wherein the reactive particle comprises zinc.

### Embodiment 25

The system of embodiment 23, wherein the reactive particle comprises gold.

### Embodiment 26

The system of any of embodiments 18-25, wherein the average diameter of the support particles is between 250 nm to 500 microns.

### Embodiment 27

The system of any of embodiments 18-25, wherein the average diameter of the support particles is between 500 nm to 10 microns.

### Embodiment 28

The system of any of embodiments 18-27, wherein the average diameter of the reactive particles is between 0.5 nm to 100 nm.

### Embodiment 29

The system of embodiment 1, wherein the average diameter of the reactive particles is between 3 nm to 20 nm.

### Embodiment 30

The system of any of embodiments 18-27, further comprising a support structure to which the composite removal particles are attached.

### Embodiment 31

The system of any of embodiments 18-30, further comprising activated carbon mercury abatement material.

### Embodiment 32

A method of decreasing the mercury content of a material, comprising the steps of contacting the material with composite removal particles, said composite removal particles comprising a support particle and a reactive particle, wherein the reactive particle of the composite removal particle combines with mercury in the material, to form a mercury-bearing composite removal particle; and removing the mercury-bearing composite removal particles from the material.

### Embodiment 33

The method of embodiment 32, wherein the material is selected from the group consisting of natural gas, liquefied natural gas, fuels, hydrocarbons, petrochemicals, and refinery streams.

### Embodiment 34

The system of embodiment 33, wherein the material is natural gas.

### Embodiment 35

The method of any of embodiments 32-34, wherein the step of contacting the material with composite removal particles comprises injecting the composite removal particles into the material.

### Embodiment 36

The method of any of embodiments 32-34, wherein the step of contacting the material with composite removal particles comprises flowing the material over a support to which the composite removal particles are attached.

### Embodiment 37

The method of any of embodiments 32-36, further comprising, before or after any step, contacting the material with activated carbon.

The disclosures of all publications, patents, patent applications and published patent applications referred to herein by an identifying citation are hereby incorporated herein by reference in their entirety. Web sites references using “World-Wide-Web” at the beginning of the Uniform Resource Locator (URL) can be accessed by replacing “World-Wide-Web” with “www.”

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

What is claimed is:

1. A system for decreasing the content of mercury in a mercury-containing flue gas stream, comprising:
composite removal particles positioned in a path of the mercury-containing flue gas stream, wherein a composite removal particle comprises a support particle and a reactive particle, wherein the reactive particle of the composite removal particle combines with mercury in the flue gas stream, to form a mercury-bearing composite removal particle; and
a trap for removal of the mercury-bearing composite removal particles, wherein the mercury content of the flue gas stream is decreased.

2. The system of claim 1, wherein the support particle comprises a material selected from the group consisting of a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite and zeolite.

3. The system of claim 2, wherein the support particle comprises silicon dioxide.

4. The system of claim 1, wherein the reactive particle comprises a material selected from the group consisting of zinc, gold, silver, tin, magnesium, lead, elemental sulfur, selenium, tellurium, platinum, and palladium.

5. The system of claim 4, wherein the reactive particle comprises zinc.

6. The system of claim 4, wherein the reactive particle comprises gold.

7. The system of claim 1, wherein the average diameter of the support particles is between 250 nm to 500 microns.

8. The system of claim 1, wherein the average diameter of the support particles is between 500 nm to 10 microns.

9. The system of claim 1, wherein the average diameter of the reactive particles is between 0.5 nm to 100 nm.

10. The system of claim 1, wherein the average diameter of the reactive particles is between 3 nm to 20 nm.

11. The system of claim 1, further comprising a support structure to which the composite removal particles are attached.

12. The system of claim 1, further comprising activated carbon mercury abatement material positioned in the path of the flue gas stream.

13. A method of decreasing the mercury content of mercury-containing flue gas stream, comprising the steps of:
contacting the flue gas stream with composite removal particles, said composite removal particles comprising a support particle and a reactive particle, wherein the reactive particle of the composite removal particle combines with mercury in the flue gas, to form a mercury-bearing composite removal particle; and
removing the mercury-bearing composite removal particles from the flue gas.

14. The method of claim 13, wherein the step of contacting the flue gas with composite removal particles comprises injecting the composite removal particles into the flue gas.

15. The method of claim 13, wherein the step of contacting the flue gas with composite removal particles comprises flowing the flue gas over a support to which the composite removal particles are attached.

16. The method of claim 13, further comprising, before or after any step, contacting the flue gas with activated carbon.

17. A composition comprising concrete or a concrete mix, said concrete or concrete mix further comprising mercury-bearing composite removal particles.

18. A system for decreasing the content of mercury in a material, comprising:
composite removal particles, wherein a composite removal particle comprises a support particle and a reactive particle, wherein the reactive particle of the composite removal particle combines with mercury in the material, to form a mercury-bearing composite removal particle; and
a trap for removal of the mercury-bearing composite removal particles, whereby the mercury content of the material is decreased.

19. The system of claim 18, wherein the material is selected from the group consisting of natural gas, liquefied natural gas, fuels, hydrocarbons, petrochemicals, and refinery streams.

20. The system of claim 19, wherein the material is natural gas.

21. The system of claim 18, wherein the support particle comprises a material selected from the group consisting of a metal oxide, iron (II) oxide, iron (III) oxide, a mixed iron oxide, copper oxide, titanium dioxide, aluminum oxide, manganese oxide, cerium oxide, molybdenum oxide, a metal nitride, titanium nitride, molybdenum nitride, a metal carbide, iron carbide, titanium carbide, molybdenum carbide, carbon, an inorganic oxide, an inorganic nitride, silicon dioxide, silicon carbide, a mixed metal oxide-hydroxide, a ceramic, boehmite and zeolite.

22. The system of claim 21, wherein the support particle comprises silicon dioxide.

23. The system of claim 18, wherein the reactive particle comprises a material selected from the group consisting of zinc, gold, silver, tin, magnesium, lead, elemental sulfur, selenium, tellurium, platinum, and palladium.

24. The system of claim 23, wherein the reactive particle comprises zinc.

25. The system of claim 23, wherein the reactive particle comprises gold.

26. The system of claim 18, wherein the average diameter of the support particles is between 250 nm to 500 microns.

27. The system of claim 18, wherein the average diameter of the support particles is between 500 nm to 10 microns.

28. The system of claim 18, wherein the average diameter of the reactive particles is between 0.5 nm to 100 nm.

29. The system of claim 1, wherein the average diameter of the reactive particles is between 3 nm to 20 nm.

30. The system of claim 1, further comprising a support structure to which the composite removal particles are attached.

31. The system of claim 1, further comprising activated carbon mercury abatement material.

32. A method of decreasing the mercury content of a material, comprising the steps of:
contacting the material with composite removal particles, said composite removal particles comprising a support particle and a reactive particle, wherein the reactive particle of the composite removal particle combines with mercury in the material, to form a mercury-bearing composite removal particle; and
removing the mercury-bearing composite removal particles from the material.

33. The method of claim 32, wherein the material is selected from the group consisting of natural gas, liquefied natural gas, fuels, hydrocarbons, petrochemicals, and refinery streams.

34. The system of claim 33, wherein the material is natural gas.

35. The method of claim 32, wherein the step of contacting the material with composite removal particles comprises injecting the composite removal particles into the material.

36. The method of claim 32, wherein the step of contacting the material with composite removal particles comprises flowing the material over a support to which the composite removal particles are attached.

37. The method of claim 32, further comprising, before or after any step, contacting the material with activated carbon.
